(19) United States
(12) Patent Application Publication
(10) Pub. No.: US 2011/0136163 A1
(43) Pub. Date: Jun. 9, 2011

(54) Method of Toxicological Assessment

(75) Inventors: Dmitri Papkovsky, County Cork (IE); Grzegorz Jasionek, Cork (IE); Alexander Zhdanov, Cork (IE)

(73) Assignee: UNIVERSITY COLLEGE CORK, NATIONAL UNIVERSITY OF IRELAND, CORK, Cork (IE)

(21) Appl. No.: 12/997,073

(22) PCT Filed: Jun. 25, 2008
(86) PCT No.: PCT/IE08/00066

§ 371 Date: Jan. 24, 2011

(51) Int. Cl. G01N 33/50; C12Q 1/02; G01N 27/26

(52) U.S. Cl. 435/29; 205/778

(57) ABSTRACT

A method of assessing toxicity of a candidate agent to a sample of cells comprises the steps of providing a sample of cells, exposing the cells to the candidate agent for a suitable period of time, assaying the cells to measure data for at least one parameter of cellular function; and correlating the measured data of the at least one parameter of cellular function with toxicity, wherein the step of exposing the cells to the candidate agent is carried out in the presence of a reagent capable of facilitating transport of the candidate agent into the cell. The transport reagent may be an endocytosis, pinocytosis inducing agent, a peptide, or a liposome. The at least one parameter of cellular function may be selected from the group consisting of: cell viability; proliferation rate; membrane integrity; and a metabolic parameter. Also described is a method of generating a toxicity signature for a candidate agent comprising the step of carrying out the method of the invention for a plurality of cellular function parameters, and compiling the measured data for each of the cellular function parameters to provide a toxicity signature.

### INTRODUCTION

The invention relates to a method of assessing toxicity of a candidate agent to a sample of cells. It finds application in many areas such as biomedical and environmental science, monitoring of water, soil and air samples, risk assessment for chemicals, safety assessment of new drugs, food safety, and general cell biology.

### BACKGROUND TO THE INVENTION

Toxicological assessment of chemical, biological and environmental samples is important for many fields. Traditionally this has been conducted using relevant in vivo models such as laboratory animals (mice, rats, guinea pigs), fish, higher organisms and humans. Such methods include cruel mortality tests to determine lethal thresholds (LD50), post-mortem histological and histochemical assessment of animal organs, tissues and individual cells for the damage caused by the toxicant. Factors such as toxicant type, dose, exposure time, administration route are usually analysed and correlated with the toxicological impact. These techniques are lengthy, expensive, have low sample throughput and ethical issues associated with their use. There is a trend to minimise the use of higher animals and replace them with alternative animal models and methods of toxicological assessment (see e.g. O'Mahony F C at al.—Environ. Sci. Technol. 2005, 39, 5010-5014). New toxicological methods that would allow rapid processing of large numbers of samples and toxicants which must undergo such tests and that produce adequate results are highly needed.

To address these challenges, a variety of alternative in vitro models are being actively developed and deployed in different areas of toxicology and in specific applications. A number of assays using simple invertebrate and vertebrate organisms such as Daphnia, Artemia salina, zebrafish, Caenorhabditis elegans is available (e.g. O'Mahony F C at al.—Environ. Sci. Technol. 2005, v.39, p. 5010), however, toxicological data generated with these models is difficult to apply to higher animals and humans. On the other hand, in vitro assays using immortalised lines of mammalian cells, including those derived from primary human and animal cells from different organs and tissues, are gaining wider use in the assessment of various chemicals, drugs and other samples, as they can produce more relevant toxicological data. Such cell models allow for simple screening micro-assays to be conducted using standard plates (96/384/1536-well microtitter plates), liquid handling (micropipettes and robotic dispensing) and detection (plate readers, imagers) equipment. Different readout parameters are used in such toxicity assays based on cultured mammalian cells. Cytotoxicity assays based on the assessment of general viability, integrity, proliferation rate of the cells, e.g. by monitoring of ³H-thymidine inclusion, MTT/alamar blue conversion, LDH release, ATP, NAD(P)H assays (Hynes J et al.—J. Immunol. Methods 2005, v.306, p. 193), are simple, inexpensive and robust. However, low specificity of these assays and end-point detection limit their use, also they do not inform on particular mechanisms of toxicity and/or targets within the cell. These assays are being replaced by the assays measuring more specific parameters of cellular function such as apoptotic markers (caspases, DNA fragmentation, Annexin V), gene and protein markers, cell signalling pathways and cascades, calcium fluctuations, metabolic and mitochondrial function biomarkers (mitochondrial membrane potential, oxygen consumption, red-ox state, extracellular acidification) (Hynes J et al.—J. Immunol. Methods 2005, v.306, p. 193). The latter group of markers of cell metabolism and mitochondrial function are gaining increasing interest, as they serve as very specific and sensitive indicators of cellular dysfunction and provide measurable changes at very low, sublethal concentrations of toxicants (Papkovsky D B et al.—Expert Opin. Drug Metab. Toxicol. 2006, v.2, p. 313).

At the same time, the use of mammalian cell lines in in vitro toxicity testing has a number of limitations. Compared to corresponding primary cells and tissues, in cultured cell lines many important features and functions are often altered, deregulated or lost. Even freshly isolated primary cells maintained in culture for short periods of time are known to undergo morphological transformations and changes in gene and protein expression. For example, this can happen with cell surface receptors and molecules that mediate transport of metabolites or particular toxicants from the site of entry in the organism (i.e. from extracellular environment) into the cell. For many toxicants molecular targets, which initiate or mediate their toxic effects, are located inside the cell or in specific sub-cellular compartments. Transport mechanisms and delivery pathways into the cells are therefore very important for such toxicants (Dawson J F, Holmes C F. Front Biosci. 1999; 4:D646-58). They are often rather specific and associated with a particular type of receptor present only in certain tissues or cell populations. Therefore, if the chosen cell model appears to lack the required receptors and/or transport mechanisms, the corresponding toxicity assay will not be able to provide adequate assessment for such toxicant. Furthermore, it may lead to erroneous or misleading results (e.g. immeasurable or reduced toxicity in vitro for a compound which is highly toxic in vivo).

For many individual toxicants and groups of compounds having similar structure and properties the main transport mechanisms into the cell, targets within the cell and mechanisms of toxicological impact have been identified and well studied (Dawson, R. M.—Toxicon 1998 36, pp. 953-962). At the same time, it is always possible for a toxicant to have multiple targets within the cell and/or several different mechanisms of action. Using the established toxicological approaches, cell models and in vitro assays, these pathways are rather difficult to investigate. Also for certain in vivo models, disease states and groups of toxicants, production of corresponding cell lines that would provide adequate toxicological assessment is problematic.

As a result, new assays and approaches are required, to ensure more reliable toxicological assessment of both known and new toxicants and of various medical, pharmaceutical and environmental samples. The detection of low levels of toxicity, low doses of toxicants and/or prolonged exposure to organisms, better prediction of toxicological risk and hazard to higher organisms and humans, more detailed assessment of mechanisms of toxicity, identification of molecular and cellular targets is high on the agenda. The situation is even more dramatic with the assessment of complex mixtures of toxicants and samples having complex or undefined composition (e.g. environmental samples).

Current invention addresses many of these problems and practical needs for new, more efficient and adequate in vitro toxicity assays. It provides new efficient means for simple, sensitive, high throughput toxicological assessment of various samples and toxicants using mammalian cells (both primary cells and cultured cell lines), and also helps elaborate modes of toxicity and potential hazard to higher organisms and humans of such samples and toxicants.

### STATEMENTS OF INVENTION

According to the invention, there is provided a method of assessing toxicity of a candidate agent to a sample of cells, the method comprising the steps of:
a) providing a sample of cells;
b) exposing the cells to the candidate agent for a suitable period of time;
c) assaying the cells to measure data for at least one parameter of cellular function; and
d) correlating the measured data of the at least one parameter of cellular function with toxicity,
   wherein the step of exposing the cells to the candidate agent is carried out in the presence of a reagent capable of facilitating transport of the candidate agent into the cell.

In one embodiment, the transport reagent comprises a liposome, typically a cationic liposome. This is especially suitable when the candidate agent has an anionic character. Examples of such liposomes will be known to those skilled in the field of drug delivery, and would include liposomal formulations based on phosphatidyl choline, lecithin, and other lipids, and morpholines. Specific examples of liposomal reagents are sold under the trade name Lipofectamine2000 (Invitrogen, U.S. Pat. No. 5,334,761) and the ESCORT family (Sigma).

In one embodiment, the transport reagent comprises an endocytosis or pinocytosis inducing agent, typically a transfection reagent. ENDOPORTER (Gene Tools) is a good example of a endocytosis or pinocytosis inducing agent that functions in a transfection manner (Summerton J E. Ann N Y Acad Sci. 2005 November; 1058:62-75).

Suitably, the transport reagent is peptide, a peptide analog, or a peptide mimetic, like ENDOPORTER (Gene Tools) (Summerton J E. Ann N Y Acad Sci. 2005 November; 1058:62-75) and DeliverX (Panomics) (Deshayes S et al. Biochem Biophys Acta. 2004. 1667(2):141-7) and CHARIOT (Active Motif).

A further suitable transport reagent is the reagent sold under the trade name FUGENE (Roche). Other suitable reagents suitable for transporting candidate agents into a eukaryotic cell will be well known to those skilled in the art.

Ideally, the transport reagent is capable of transporting a plurality of different candidate agents in a relatively non-specific manner.

Typically, the transport reagent is capable of transporting candidate agents into a plurality of different cells.

Suitably, the at least one parameter of cellular function is selected from the group consisting of: cell viability; proliferation rate; membrane integrity; and a metabolic parameter. Typically, the metabolic parameter is selected from the group consisting of: oxygen consumption rate; the levels of cellular ATP, NADH; mitochondrial membrane potential, intracellular Ca²⁺; apoptotic markers; level of extracellular acidification; and level of signalling or neurotransmitter markers. In a preferred embodiment, several parameters of cellular function are analysed in parallel.

In one embodiment of the invention, the step of measuring data of the at least one parameter of cellular function is an end-point measurement. In a preferred embodiment, during the assay step, data of at least one parameter of cellular function is monitored continuously.

The data for the at least one parameter of cellular function may be measured by any suitable means. For example, the data may be measured by optical (fluorescence, absorbance, bio- or chemiluminescence), electrochemical, magnetic or other means.

In one embodiment of the invention, the measured data for the at least one parameter of cellular function is correlated with toxicity by comparing the measured data with reference data for the parameter of cellular function. This reference data is generally obtained using the same cells and conditions as the test assay, with the exception that the candidate agent is not included. In other words, the cells are treated with transport reagent but not the candidate agent.

Typically, the sample of cells is selected from the group consisting of: an in-vitro cell culture; artificially transformed cells; primary cells from higher animals and humans isolated from different tissues and organs; and a tissue explant.

In a preferred embodiment of the invention, the sample of cells is exposed to different doses of the candidate agent. Alternatively, or additionally, the sample of cells may be treated with the candidate agent at a plurality of temperatures. Alternatively, or additionally, the sample of cells may be treated with the candidate agent in a plurality of media. Alternatively, or additionally, the sample of cells may be treated with the candidate agent for a plurality of exposure times.

In an embodiment in which multiple assays are to be carried out, the method of the invention, in particular steps (b) and (c), are carried out in the wells of a microtitre plate.

The methods of the invention are especially suitable for assessing the toxic load of marine toxins, especially microcystins, food toxins, environmental toxicants and particulate matter. The methods of the invention are especially suitable for assessing the toxic load of toxins which are cell membrane impermeable and/or require the presence of a specific cross-membrane transport mechanism.

The invention also relates to a method of generating a toxicity signature for a candidate agent comprising the step of carrying out the method of the invention for a plurality of cellular function parameters, and compiling the measured data for each of the cellular function parameters to provide a toxicity signature. Generally, the toxicity signature is cell-specific.

The invention also provides a method of detecting toxic contamination of a cell comprising a step of assaying the cell to measure data of a plurality of parameters of cellular function, compiling the measured data to provide a test toxicity signature, and comparing the test toxicity signature with known toxicity signatures to identify the source of the toxic contamination.

### BRIEF DESCRIPTION OF THE FIGURES

FIG. 1. Alterations in oxygen consumption in primary hepatocytes, HepG2 and Jurkat cells caused by MCLR with and without the addition of the loading reagent (Endoporter).

FIG. 2. Western blot analysis of HepG2 cells treated with MCLR in the presence of EndoPorter for 24 h, primary antibodies used were anti-phospho-serine. Blot analysis show dependence of protein phosphorylation on MCLR concentration.

FIG. 3. Oxygen consumption rates of Jurkat T and HepG2 cells after incubation with Aroclor alone for 24 h (◯ Jurkat, □ HepG2), with Aroclor and EndoPorter for 24 h (• Jurkat, HepG2).

### DETAILED DESCRIPTION OF INVENTION

The invention provides a new approach to the assessment of toxicological impact of chemicals and other samples on live organisms, particularly higher animals and humans. It provides a new format of toxicity assays using live mammalian cells as a model and new assay methodology which effectively overcomes the problems associated with transport of toxic specie into the cell where they can exhibit their toxic action. This is particularly important for toxicants whose main targets are located inside the cell and for which transport into the cells involves relatively specific or even unknown mechanism(s). Such transport can potentially be a critical factor in in-vitro cell-based assays with these toxicants.

The invention and corresponding cell-based assays are aimed at reducing the dependence of the outcome of such assays from cell-specific and toxicant-specific transport into the cell. To achieve this, facilitated transport of the toxicant into the cells is incorporated in assay procedure. This is achieved by using special reagents and (bio)chemicals which promote transport of the toxicants of interest into the cell. Such reagents generally lack cell specificity, are non-toxic to the cells and not interfere with cellular function. These reagents are added to the medium in which the cell-based toxicity assay is carried out, so that they act on the cells in a predictable manner stimulating transport of the toxicant into the cells.

A number of such reagents which can facilitate transport of toxicants into mammalian cells and which satisfy other requirements of corresponding toxicity assays have been identified and demonstrated in this invention. For example, cationic liposomal reagents were found to be efficient, particularly with toxicants of anionic nature. Examples of such reagents include liposomal formulations based on phosphatidylcholin and some other analogs, morpholines, and commercial reagents such as Escort family (Sigma, http://www.sigmaaldrich.com/sigma/datasheet/16037dat.pdf). These reagents have low intrinsic toxicity and work with different cells. Although they were originally developed for delivery of nucleic acids (DNA, RNA) into the cells, we have shown that they are highly advantageous in cell based toxicity assays. On the other hand, transport with these reagents requires binding of cargo to the liposomal vesicles which then fuse with cell. This mechanism may not work for certain chemicals and in certain media (e.g. protein-free medium is often required) (Summerton J E—Ann. N-Y. Acad. Sci., 2005, 1058: 62-75).

The non-liposomal transfection reagents were also found to be efficient for the invention, particularly those which activate the process of endocytosis by the cells. In the presence of such reagents, the cells start to actively internalise fragments of the cell membrane and microdroplets of extracellular medium. Such vesicles, which contain extracellular medium and its components, subsequently release their content into the cell. Such reagents and mechanisms enable facilitated transport of various components present in assay medium (Summerton J E—Ann. N-Y. Acad. Sci., 2005, 1058: 62-75; U.S. Pat. No. 7,374,778), therefore, they are applicable to a broad range of toxicants. A number of other reagents and methodologies known to facilitate transport of toxicants into the cells can also be used in the invention.

We also demonstrated that a range of existing cell-based toxicity assays can be easily adapted to the new methodology, by simple modification(s) of their general procedures. In such assay, the cells prepared for the assessment are exposed to the toxicant at a certain dose and for a certain period of time under the defined conditions (medium, temperature, etc.). During or after such an exposure to the toxicant, which is now conducted in the presence of transporting reagent, changes in cellular function are analysed with an appropriate probe, biomarker or bioassay of cellular function. The effect of the toxicant on the cells is thus determined, either qualitatively or quantitatively. Samples containing the same cells exposed to the transporting reagent without toxicant are often incorporated in such assays, and the effects of toxicants are related to these samples (controls).

We have shown that this methodology of toxicity assays can operate with a range of established lines of mammalian cell and also with new cell lines, primary cells and tissues. It allows rapid and simple processing of large number of samples using conventional microtitter plates (96/384/1536-well), producing relevant and information-rich data on the action of toxicants on live cells. The invention demonstrates that this approach is applicable to different types of toxicants, including small molecules, macromolecules, nano- and microparticles. It allows easy integration in the existing cell based assays providing advantages and new opportunities. As illustrated in the examples below, a significant improvement in assay sensitivity with respect to particular toxicants, better adequacy of toxicological assessment, the possibility to replace more complex primary cell models with conventional cell line models were achieved (see e.g. microcystins).

Furthermore, the new approach was seen to be particularly efficient in the assays which rely on the monitoring of key parameters of metabolism, mitochondrial function and bioenergetic status of the cell. Parameters, such as mitochondrial membrane potential, oxygen consumption rate and intracellular oxygen concentration, NAD(P)H:NAD+ balance, ATP, intracellular calcium, fluxes of principal ions, intracellular pH, extracellular acidification rate, are known to be sensitive and early markers of cellular dysfunction. Such assays often rely on fluorescence-based probes (endogenous or genetically encoded) and detection formats, which are minimally invasive to the cells and which provide high sensitivity, selectivity, high throughput screening and high content imaging capabilities. They also provide continuous, real-time multi-parametric readout of corresponding parameters, which is highly advantageous for toxicological assessment. The introduction of new biomarkers of toxicity and development of enabling technologies for toxicological assessment (such as live cell fluorescent imaging) can also be integrated with such assays.

Furthermore, different parameters and corresponding cell based toxicity assays mentioned above provide a different insight into cellular function and inform on the action of the toxicant on test cells in different ways. They can be applied to toxicological assessment of compounds either individually, or in combination using arrays of assays. The latter approach is more advantageous for the determination of specific targets within the cell and fine mechanisms of toxic action. It is also useful for identifying particular assays which provide optimal sensitivity, selectivity and convenience for the detection of given toxicant or groups of toxicants. Basic setup and experimental details for measuring these parameters of cellular function and conducting corresponding cytotoxicity assays are well known to specialists in the area and also described in different modifications in scientific papers, textbooks and patents. However, so far they have not been used in this new, modified format which is described in the invention.

When applying these assays in the new format described in the invention to various model toxicants, we discovered a number of important and unexpected features which are highly advantageous for general toxicology and also for certain practical applications. For example, we have shown that toxicity assays based on the measurement of cellular oxygen consumption or intracellular oxygen concentration, when performed in the new format of the invention, provide more sensitive, simple and versatile means for the assessment of important toxicants of different type, such as food and marine toxins, pesticides and PAHs (see Examples).

In particular, microcystins—strong hepatotoxins produced by algal blooms and present in contaminated marine and freshwater samples at very low concentrations. Microcystin-LR is a highly specific, cell-impermeable hepatotoxin, which is actively taken up by hepatocytes. Its transport is mediated by the family of Organic Anion Transporting Polypeptides (OATP) in the liver, which have a wide spectrum of substrates, they also transport bile salts, bile acids, steroids and various peptides across the hepatocyte cell membrane (Ding W X et al.—J Toxicol Environ Health A. 2001; 64(6): 507-19). When taken up by the liver cells, MCLR binds to protein phosphatases 1 and 2A (PP1, PP2A), thus inhibiting the phosphatase activity and disrupting the delicate phosphorylation/dephosphorylation balance within the cell. Acute poisoning with microcystins results in liver cell necrosis caused by disruption of cytoskeleton. MCLR uptake induces DNA damage in primary human and rat hepatocytes. MCLR treatment is related to elevated ROS levels and depletion of glutathione in primary hepatocytes cultures. Decrease in mitochondrial membrane potential and changes in mitochondria permeability transition were also observed (Komatsu M et al.—Toxicol Sci. 2007; 97(2): 407-16).

In conventional testing microcystins displayed measurable toxicity only with freshly isolated primary hepatocytes (e.g. from rat liver). In this case, a number of parameters of cellular function were seen to be affected at microcystin-LR concentrations of 0.5 nM and higher. At the same time, many common cell lines including HepG2 liver cells showed no detectable toxicity in such assays even at concentrations of 1-10 uM, i.e. 3-4 orders higher than those measurable with primary hepatocytes. This is in agreement with the transport mechanism described above, due to the fact that OATP transporter is absent in many cells and tissues. Also OATP is quickly lost by the primary hepatocytes (in 12-24 h), when they are cultured in vitro.

On the other hand, when facilitating transport of toxicant according to the invention by exposing the cells to microcystin-LR (e.g. in the presence of Endo-Porter reagent), strong toxicity was observed with HepG2 cells and well as with the other cell lines (Jurkat cells). These cells, which are normally insensitive to the toxin, become very sensitive in this new assay format. At the same time the new assay and reagent used do not alter much cellular function and parameter being measured.

Furthermore, when using these cell models (HepG2 or Jurkat cell line) which are easy to culture and manipulate, the sensitivity of such assays even exceeded that of the established assays performed with primary hepatocytes. As a result, the detection of very low concentrations of microcystins (0.1 nM and even lower) can be achieved in such toxicity assays based on the measurement of changes in oxygen consumption of HepG2 cells. In addition, toxic effects of microcystins on cells are detectable faster—after 3 h as opposed to 12-24 h exposure time in conventional assays. The use of more simple cell model and assay protocol allows processing of large number of samples in parallel (96/384 samples on one microplate).

The observed toxic effects on cells measured in the new assays displayed correlation with the dose and exposure time of the toxicant, producing characteristic profiles and patterns for different toxicants, thus showing specific action of different toxicants on test cells. Also the patterns of response depend on the cell type (for example, enhancement of respiration in HepG2 and inhibition in Jurkat cells). Thus, different toxicants can be distinguished based on their action on different cell types and parameters of cellular function.

Taken together, the new approach of the invention, when applied to the group of marine toxins such as microcystins and well as to a number of other toxins or groups of toxins of different type, provides a very sensitive and rapid but at the same time simple and high throughput tests to screen large panels of samples for the presence of these toxins in real environmental samples (e.g. sea and river water, food, wastewater, etc). The invention is therefore deemed very practical and finds application in many biochemical and environmental toxicological applications.

Although the approach described in the invention has been shown advantageous for many toxicants and samples of different type, it is believed that it is not universal and equally efficient for all toxicant of different kind. In this regard, it has been shown to have limited benefit with respect to the following species and toxicants (candidate agents):

1) highly hydrophobic molecules which have intrinsic tendency to accumulate in or penetrate cell membrane and rapidly accumulate into the cell. This includes organic solvents toluene, benzene, some polycyclic aromatics.
2) small molecules which can cross the membrane of different cells at significant rates (e.g. mimetics of principal biological ions, metabolites, hormones), or which are transported by abundant mechanisms.
3) particulate matter and macromolecules which are effective taken up by various cells.
4) toxicants which have their targets on cell surface (e.g. neuroxins targeting ion channels on cell membrane;
For these groups of toxicants transport into the cell is not a limiting step for their toxic action, therefore the invention is not expected to improve their toxicological assessment to the same degree as for the other toxicants described above. One can therefore recommend for each new type of toxicant to perform an initial basic evaluation (examining structure, possible modes of toxicity, other functions), and to conduct preliminary experiments, i.e. cell-based assays with and without transport reagent. Thus, in this specification, the term “candidate agent” should ideally be taken to exclude toxins for which transport into the cell is not a limiting step for their toxic action. Thus, the term should be understood to exclude the toxins described in 1) to 4) above.

At the same, time, the invention is readily applicable to many toxicants with both known and unknown mechanism of toxic action, to environmental samples and various other samples of unknown or undefined composition. For the latter, the initial toxicity data produced by the method of invention (relatively non-specific) can be further verified and investigated by the other more specific toxicological methods. At the same time, samples which demonstrate no significant toxicity in this method can be taken out from such more expensive and detailed examinations, thus saving time and resources for the positive samples.

The invention is further demonstrated with the following Examples, which are by no means limiting the scope of the invention.

### Example 1

### In Vitro Assessment of Toxicity of Microcystin-LR Using Primary Hepatocytes and Cultured Cells

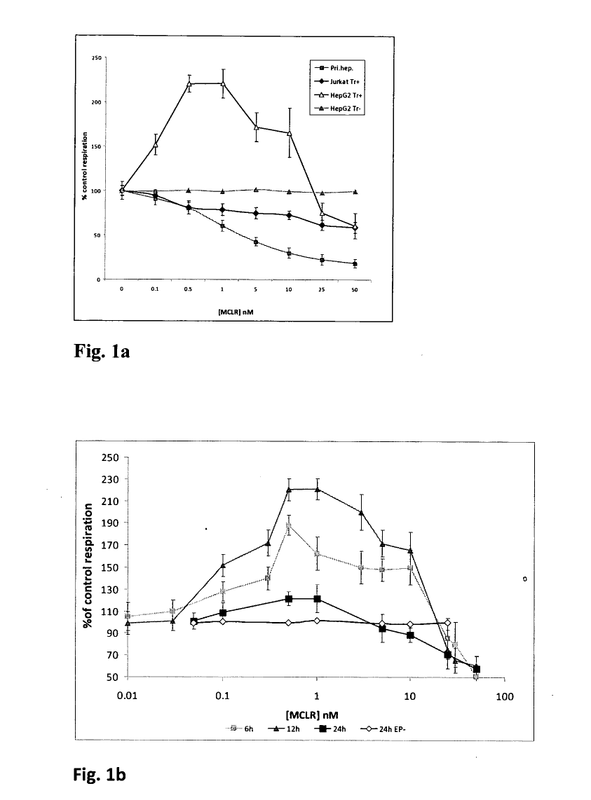

Primary rat hepatocytes were isolated from Sprague-Dawley rats by the standard two-step collagenase perfusion method, seeded on collagen-coated 96 well plates in DMEM containing 1 g/L glucose and left for 3 h to adhere at 37oC in CO2 incubator. Microcystin-LR stock in ethanol was diluted in medium, added to the test wells at the specified final concentrations and the plate was incubated for further 24 h. Controls without MCLR were also included. After the exposure, the MitoXpress oxygen probe (Luxcel Biosciences) was added at 0.15 uM to each assay well, the wells were covered with 100 ul of oil, and the plate was read at 37° C. on a fluorescence plate reader (Genios Pro, Tecan) in kinetic mode with readings in each well taken every 1 min over 60-120 min. Time-resolved fluorescent measurements were done with the following settings: excitation/emission—380/650 nm, gain—90, delay time—30 us, gate time—100 us. From the measured profiles of probe fluorescence, relative rates of oxygen consumption were calculated for each sample (as described in (Hynes J et al,—J. Immunol. Methods 306 (2005) 193-201). FIG. 1a shows dose-dependent decreases in oxygen consumption (relative to untreated cells), which are indicative of the degree of MCLR toxicity for this model. At shorter incubation periods the decrease in respiration became smaller. Similar experiments were performed with HepG2 and Jurkat cells, which showed no detectable toxicity even at much higher doses of MCLR and 24 h exposure (FIG. 1a).

When experiments were conducted with HepG2 and Jurkat cells exposed to MCLR in the presence of transport reagent Endoporter (Gene Tools), both cell lines showed marked changes in O₂ consumption. In Jurkat cells respiration decreased, whereas in HepG2 a marked increase in oxygen consumption was observed. Significant effects on both cells were seen at concentrations above 0.1 nM, with a characteristic bell-shape dose response with the peak at around 0.5 nM MCLR concentration for HepG2 cells. The response in HepG2 cells was even stronger than that observed with primary hepatocytes (without transport reagent). It was dependent on the incubation time and had the opposite sign suggesting a different mode of action or primary target compared to the ones seen in conventional assays.

### Example 2

### Elaboration of MC-LR Toxicity Revealed by the New Method

The experiments in Example 1 have revealed marked increases in oxygen consumption in HepG2 cells upon their exposure to MCLR in the presence of Endoporter. The time scale and shape of the response and changes in cellular function suggest direct action of MCLR on mitochondria. Although mitochondrial toxicity of microcystins has been reported (based on the studies with primary cells), such strong uncoupling effect was not known so far. This effect was subsequently confirmed by the analysis of O₂ consumption by isolated rat liver mitochondria in the presence of MCLR (in this case without Endoporter). Indeed, an uncoupling effect of MCLR was also observed in glutamate/malate medium but not in the other media.

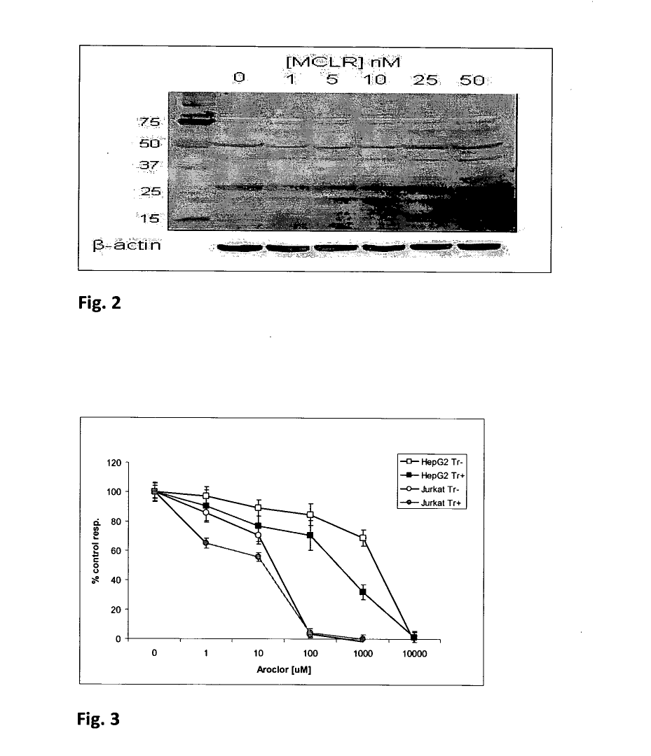

A number of other markers of cellular function were also investigated for their alterations in response to MCLR treatment of cells in the presence of transport reagent. Similar to the results of traditional assay with primary hepatocytes, in the new assay with MCLR and Endoporter reagent we observed altered protein phosphorylation/dephosphorylation in Western blots (FIG. 2.). Altogether, this data augment the results of oxygen consumption assay and the new findings about the mechanisms of toxicity of MCLR on mammalian cells. Metabolic parameters such as intracellular levels of ATP and NAD(P)H were seen unchanged. This indicates that MCLR interacts with Complex I of the electron transport chain within the cell and that the mitochondrion represents the likely primary target for MCLR, rather than cellular protein phosphotases. These experiments also demonstrate the utility of the new method in elaborating fine mechanisms of toxic action for different toxicants, and its efficiency when used in combination with the assay based on the measurement of cellular oxygen consumption—an early marker of and very sensitive assay for cellular dysfunction.

### Example 3

### The Assessment of Other Toxicants and Transporting Reagents in the New Method

A number of other transport reagents, toxicants, cell models and parameters of cellular function were examined, using the general assay format described in Example 1. Some representative data is shown in Table 1.

TABLE 1
Effects of different toxicants on test cells, with and without transport reagents.

| | | | Cellular | |
|---|---|---|---|---|
| | | Transport | Parameter | |
| Toxicant | Cell model | reagent | Assessed | Observed effect |
| MCLR | Primary | None | O2 consumption | IC50 = 2.74 nM ± 0.65 |
| | hepatocytes | | | |
| | HepG2 | None | O2 consumption | No effect at 10 μM |
| | | Endoporter | O2 consumption | Strong Uncoupling |
| | Jurkat | None | O2 consumption | No effect at 10 μM |
| | | Escort III | O2 consumption | EC50 = 4.85 nM ± 1.19 |
| | | Endoporter | O2 consumption | EC50 = 22.65 nM ± 2.89 |
| | Jurkat | None | ATP content | No effect at 10 μM |
| | | Endoporter | ATP content | Dose dependent decrease |
| Arochlor | Jurkat | None | O2 consumption | EC50 = 15.9 uM ± 3.15 |
| (pesticide) | | Endoporter | O2 consumption | EC50 = 9.88 uM ± 1.39 |
| | HepG2 | None | O2 consumption | EC50 = 1365 uM ± 90 |
| | | Endoporter | O2 consumption | EC50 = 363.4 uM ± 60.6 |
| Aflatoxin B1 | HepG2 | None | O2 consumption | EC50 = 2.31 uM ± 0.68 |
| (food toxin) | | Endoporter | O2 consumption | EC50 = 27.9 uM ± 6.1 |
| | Jurkat | None | O2 consumption | EC50 = 41.55 uM ± 13.7 |
| | | Endoporter | O2 consumption | EC50 = 8.18 uM ± 1.57 |

As expected, in many cases (but not all) the new cell-based toxicity assay incorporating transport reagent produced improved sensitivity. Thus, Aroclor belongs to polychlorinated biphenyls—compounds which are known to inhibit mitochondrial ATPase activity of different cells, showed increased toxicity with Jurkat cells, so as aflatoxin B1.

In addition, using fluorescently labelled protein (Pt-coproporphyrin conjugated to serum albumin) instead of the toxicant, transporting efficiency of several commercial transfection reagents with respect to different cells lines was evaluated. Results are shown in Table 2.

TABLE 2
Comparison of transporting efficiency of different
reagents using a fluorescent tracer.

| | | | Phosphorescent |
|---|---|---|---|
| Cell Line | Transport Reagent, Mechanism | Medium, Time | signal, cps |
| HepG2 | Fugene (Roche) - undisclosed | Serum-free, 24 h | 30,000 |
| Liver cells | mixture | | |
| | Lipofectamine (Invitrogen) - | Serum-free, 24 h | 7,000 |
| | liposomal | | |
| | Chariot (Active Motif) - peptide | Serum-free, 24 h | <2,000 |
| | Endoporter (Gene Tools) - | 5% serum, 24 h | 10,000 |
| | peptide | | |
| | Escort III (Sigma) - liposomal | Serum-free | 4,000 |
| | | medium, 24 h | |
| PC12 | Fugene (Roche) - undefined | Serum-free, 24 h | 35,000 |
| neurosecretory | mixture | | |
| cells | Lipofectamine (Invitrogen) - | Serum-free, 24 h | 1,000 |
| | liposomal | | |
| | Endo-Porter (Gene Tools) - | 5% Serum, 24 h | 50,000 |
| | peptide | | |
| | Escort III (Sigma) - liposomal | Serum-free, 24 h | 5,000 |

Such experiments can be used for optimising the type of transport reagent for toxicity assays with a particular cell line.

### Example 4

### The Analysis of Water Samples for the Contamination with Marine Toxins (Microcystins)

A panel of water samples collected from different sites contaminated with algal blooms or suspected for contamination with marine toxins were assessed by the new method. The cell-based in vitro assay was prepared and executed in standard 96-well plates according to the general procedure described in Example 1. HepG2 as cell model, Endoporter as transport reagent, 6 h exposure to the toxicant or unknown sample, MCLR as positive control and clean water as negative control were used. Water samples were diluted with assay medium (usually 1:4-1:10, to bring the osmolarity to the level tolerated by the cells) and these solutions were added to the wells with cultured cells. A number of wells were used as negative controls (cells, Endoporter and clean water), and some were exposed to MCLR at concentrations 0.03, 0.1, 3.0 and 10 nM (positive controls or standards for the calibration). Repeats were included as required (usually each samples was analysed in triplicates). Following the exposure of the cells, addition of O2 probe and measurement of O2 consumption, each assay well/sample was analysed for the changes in O2 consumption with respect to the cells without toxicants added. Samples which altered the respiration of test cells were defined as positive. Samples that showed no measurable effect on respiration were defined as negative. For positive samples the level of contamination with marine toxins was determined using the calibration produced with clean water spiked with MCLR standards. Marine toxins in the water samples were also analysed by standard method (LC-MS), these results showed good correlation with the new method.

The invention is not limited to the embodiment hereinbefore described which may be varied in both construction, detail, and process step without departing from the spirit of the invention.

1. A method of assessing toxicity of a toxin to a sample of mammalian cells comprising the steps of:
a) providing a sample of mammalian cells;
b) exposing the cells to the toxin for a suitable period of time;
c) assaying the cells to measure data for at least one parameter of cellular function; and
d) correlating the measured data of the at least one parameter of cellular function with toxicity,
wherein the step of exposing the cells to the toxin is carried out in the presence of an endocytosis or pinocytosis inducing agent, and wherein the toxin is cell membrane impermeable or a toxin for which transport into the mammalian cell is a limiting step for its toxic action.

2. A method as claimed in claim 1 in which the endocytosis or pinocytosis inducing agent is Endoporter.

3. A method as claimed in claim 1 in which the at least one parameter of cellular function is selected from the group consisting of: cell viability; proliferation rate; membrane integrity; and a metabolic parameter.

4. A method as claimed in claim 3 in which the metabolic parameter is selected from the group consisting of: oxygen consumption rate; the levels of cellular ATP, NADH; mitochondrial membrane potential, intracellular Ca²⁺; apoptotic markers; level of extracellular acidification; and level of signalling or neurotransmitter markers.

5. A method as claimed in claim 1 in which the step of measuring data of the at least one parameter of cellular function is an end-point measurement, and/or in which during the assay step data of at least one parameter of cellular function is monitored continuously.

6. A method as claimed in claim 1 in which data for the at least one parameter of cellular function is measured by optical (fluorescence, absorbance, bio- or chemiluminescence), electrochemical, magnetic or other means.

7. A method as claimed claim 1 in which the measured data for the at least one parameter of cellular function is correlated with toxicity by comparing the measured data with reference data for the parameter of cellular function obtained from the cells treated with the endocytosis or pinocytosis inducing agent but not the toxin.

8. A method as claimed in claim 1 in which the sample of cells is selected from the group consisting of: an in-vitro cell culture; an isolate of primary cells from a higher animal or human; an artificially transformed cell line; and a tissue explant.

9. A method as claimed in claim 1 in which the sample of cells is treated with different doses of the toxin, and/or in which the sample of cells is treated with the toxin at a plurality of temperatures.

10. A method as claimed in claim 1 in which the sample of cells is treated with the toxin in a plurality of media and/or in which the sample of cells is treated with the toxin for a plurality of exposure times.

11. A method as claimed in claim 1 in which the toxicity of the toxin on the cells is correlated with its dose.

12. A method as claimed in claim 1 in which steps (b) and (c) are carried out in the wells of a microtitre plate.

13. A method of generating a toxicity signature for a toxin comprising the step of (a) carrying out the method of claim 1 for a plurality of cellular function parameters, and compiling the measured data for each of the cellular function parameters to provide a toxicity signature; and/or carrying out the method of claim 1 for a plurality of different cell types, and compiling the measured data for the cellular function parameters to provide a toxicity signature.

14. A method of detecting contamination of a sample by a toxin comprising a step of assaying the effects of the sample on test mammalian cells according to claim 1, and compiling the measured data to provide an estimate of sample toxicity.

15. A method of detecting contamination of a sample by a toxin comprising a step of assaying the effects of the sample on test mammalian cells according to claim 1, and compiling the measured data to provide a test toxicity signature, and comparing the test toxicity signature with known toxicity signatures to identify the type of the toxic contamination.

16.-29. (canceled)

30. A method as claimed in claim 2 in which the step of measuring data of the at least one parameter of cellular function is an end-point measurement, and/or in which during the assay step data of at least one parameter of cellular function is monitored continuously.

31. A method as claimed in claim 3 in which the step of measuring data of the at least one parameter of cellular function is an end-point measurement, and/or in which during the assay step data of at least one parameter of cellular function is monitored continuously.

32. A method as claimed in claim 42 in which the step of measuring data of the at least one parameter of cellular function is an end-point measurement, and/or in which during the assay step data of at least one parameter of cellular function is monitored continuously.

33. A method as claimed in claim 2 in which data for the at least one parameter of cellular function is measured by optical (fluorescence, absorbance, bio- or chemiluminescence), electrochemical, magnetic or other means.

34. A method as claimed in claim 3 in which data for the at least one parameter of cellular function is measured by optical (fluorescence, absorbance, bio- or chemiluminescence), electrochemical, magnetic or other means.
